(19) United States
(12) Patent Application Publication
(10) Pub. No.: US 2004/0258822 A1
(43) Pub. Date: Dec. 23, 2004

(54) Chilsonated sucralose product

(76) Inventors: Shyhyuan Liao, West Windsor, NJ (US); Carolyn M. Merkel, North Haledon, NJ (US); Steven J. Catani, Athens, GA (US)

Correspondence Address: RATNERPRESTIA, P O BOX 980, VALLEY FORGE, PA, 19482-0980, US

(21) Appl. No.: 10/865,259

(22) Filed: Jun. 10, 2004

Related U.S. Application Data
(60) Provisional application No. 60/478,794, filed on Jun. 16, 2003.

(51) Int. Cl. A23G 3/30

(52) U.S. Cl. 426/548

(57) ABSTRACT

An artificial sweetener composition contains sucralose and optionally a bulking agent such as maltodextrin. The composition is in the form of granules having a size between 30 and 2000 μm and containing between 0.5 and 1.5 wt % moisture. Any process can be used for making the compositions, with one especially applicable one involving pressing a powdered feed containing the raw materials between rolls to form a densified mass. The densified mass is then broken down in size and optionally fractionated by particle size.

### FIELD OF THE INVENTION

The present invention relates to artificial sweeteners. More specifically, it relates to granular sweetener particles containing sucralose, and methods of making them that include compaction of a sucralose-containing formulation, grinding, and (optionally) screening to produce desired granule size fractions.

### BACKGROUND OF THE INVENTION

High-intensity sweeteners can provide the sweetness of sugar, with various taste qualities. Because they are many times sweeter than sugar, however, much less of the sweetener is required to replace the sugar. High-intensity sweeteners have a wide range of chemically distinct structures and hence possess varying properties.

In order for a high-intensity sweetener to be conveniently used for dry blending and tableting, several criteria should be met. These include good flow properties, little dust formation during processing, absence of static electric problems, good mechanical strength, and good stability.

Sucralose (1,6-dichloro-1,6-dideoxy-β-D-fructofuranosyl-4-chloro-4-deoxy-α-D-galactopyranoside) is a high-intensity sweetener made by the selective chlorination of sucrose. Sucralose is a white, crystalline, nonhygroscopic, free-flowing powder in its pure form. It is highly soluble in water, ethanol, and methanol and has a negligible effect on the pH of solutions.

Although the stability of dry sucralose is generally good as compared to other high-intensity sweeteners, its stability can be affected by temperature, time, moisture, and packaging conditions. Initial decomposition of the dry sucralose at high temperature results in discoloration. As used herein, the term “dry” when referring to sucralose means a solid form as opposed to a dissolved form, unless the moisture content of the sucralose is specifically being discussed. Discoloration of dry sucralose is accompanied by a very small release of hydrogen chloride, which can be detected by measuring pH changes in an aqueous solution of sucralose. Changes in color, pH, and absorbency characteristics due to decomposition occur before any significant loss of sucralose can be measured.

Reducing the particle size of sucralose by micronization (milling to produce small particles) has been shown to improve stability. However, micronization of sucralose brings several practical problems. First, the micronized product is very fine (approximately 95% of the particles are less than 10 μm in size) such that the particles often stick together and become clumpy, thus making the product less free-flowing. Further, dusting often occurs during processing, because the micronized product is so fine, and significant amounts of sucralose may be lost. Finally, the micronized particles have weak mechanical strength, tend to break apart, and are therefore not ideal for blending or tableting. A sucralose composition having both good handling and stability performance would be of value in commercial applications.

### SUMMARY OF THE INVENTION

In one aspect, the invention provides a composition comprising granules comprising sucralose, wherein the granules are between 30 and 2000 μm in size and have a moisture content between 0.5 and 1.5 wt %.

In another aspect, the invention provides a method for obtaining granules comprising sucralose. The method comprises the steps of:

A) providing a feed comprising sucralose, the feed having a starting moisture content selected such that the granules have a final moisture content between 0.5 and 1.5 wt %;

B) compacting the feed to form a compacted material; and

C) grinding the compacted material to form the granules.

In still another aspect, the invention provides granules, made by the above method, comprising sucralose.

It is to be understood that both the foregoing general description and the following detailed description are exemplary, but are not restrictive, of the invention.

### DETAILED DESCRIPTION OF THE INVENTION

The present invention discloses sucralose-containing granules and a process for making such granules. The granules have good flow properties, low dust formation, good mechanical strength, little tendency to generate static electric charge, and good stability. Specifically, the objectives of this invention are: 1) to provide a more stable dry sucralose material which is useful for dry blending and tableting; and 2) to provide a process for obtaining such a highly desirable form of dry sucralose with the ability to isolate specifically desired particle sizes.

The granular product of the invention involves compaction of sucralose-containing compositions, and this may be accomplished using any known compaction technique. Suitable techniques include roller compaction, tableting, slugging, ram extrusion, plunger pressing, roller briquetting, reciprocating piston processing, die pressing, and pelleting. One especially useful method of compaction is roller compaction, and a particularly effective form of this is used in a process known in the art as “chilsonation” using roller compaction equipment such as that available from The Fitzpatrick Company, Elmhurst, Ill. As used herein, the term “chilsonation” and variations thereof refers to a dry granulation process in which a particulate material is compacted under high pressure (typically between about 1000 and 2000 psi) bypassing between two rollers, which may optionally be textured (e.g. grooved, waffled, etc.) to produce a compacted, densified material (the “compact”). The compact may be of any thickness, but in one exemplary embodiment the system is adjusted to provide a sheet of about 0.1 to 0.3 inches in thickness exiting the rollers. The product exiting the rollers typically has a density between about 1.46 and about 1.57 g/cc, although this is not necessary to the practice of the invention. It is advantageous to use a roller that has a polytetrafluoroethylene or other non-stick coating, and to shield the roller from sources of heat such as the drive motor, to help prevent sticking of the composition during the compacting step. It may also be helpful to apply a vacuum to the feed in the screw portion of the chilsonator that feeds the compacting rollers. Use of vacuum may improve the handling and feeding of the sucralose through the screw into the rollers, particularly if the feed includes a large amount of fines that tend to become suspended in the air.

The material being fed to the rollers may be raw sucralose taken directly from the centrifuge (“wet cake” material, which has a moisture content of about 3-5 wt %), or the sucralose may first be dried to the desired level. In another embodiment, a mixture of sucraloses having different moisture contents may be used, for example a mixture of wet cake material and neat material. As used herein, the term “neat sucralose” refers to wet cake material that has been dried to a moisture content below 0.1 wt %. It has been found that many of the performance properties of the granulated composition benefit from the presence of a certain amount of moisture in them, including good flowability, mechanical strength, and storage stability. Typically, the moisture content of sucralose feed being compacted according to some embodiments of the invention is greater than 0.5 wt %, more typically greater than 0.7 wt %, and less than 3 wt %, more typically less than 1.5 wt %. Generally, the moisture content of the granules is somewhat lower than that of the feed, perhaps due to evaporation during the compaction process and/or during conveying of the feed to the chilsonator or other compaction device. Typically, the moisture content of sucralose granules according to some embodiments of the invention is greater than 0.5 wt %, and less than 1.5 wt %, more typically less than 1.2 wt %.

In one embodiment of the invention, wet cake from the sucralose manufacturing process is dried until a desired moisture content is reached, and then the material is used directly. This may provide an energy-efficient way of achieving a desired moisture content. Although this material (or the dry portion of a moist/dry mixture) may be micronized if desired before feeding to the chilsonator, this is not necessary. Thus a step may be saved, and losses due to dusting of micronized sucralose may also be reduced.

The compact may take any form that can be subjected to subsequent size reduction. Suitable forms include flakes, chips, briquettes, chunks, and pellets. The shape and appearance of the compact will clearly depend upon the shape and surface characteristics of the equipment used to perform the compacting step. In this regard, compact may appear smooth, corrugated, fluted, or pillow-pocketed. The actual size of the compact will also be dependent upon the type of equipment and operation parameters employed during compaction. It will be appreciated that optimization of feed rates, moisture content of the feed, roller pressures, roller rotation rate, and other parameters may be needed in order to arrive at a product having a particular set of desired properties, and that these parameters will vary according to that set of properties. Some degree of routine optimization may therefore be required, but such optimization is within the ability of the person of ordinary skill in the art.

Once the compact is formed, it is then ground or broken apart using any known technique. Typically a mill is used, and the grinding or breaking of the compact is accomplished in two basic steps, namely, a coarse grinding step and a subsequent milling step. The grinding process can be accomplished in a single step or it may be accomplished using a series of steps and a variety of mill opening or pore sizes. The specifics of the mill (i.e., type of blades, rotor speed) can be adjusted to create the desired particle size.

Once ground, the sucralose granules are run through a final size fractionation step in order to obtain granules of a particular desired particle size. This step can be accomplished using any known technique, and may include for example air classification and screening. Typically a screening method is used, using standard screens and sifters and, and more typically using a sizing-sorting screening machine such as is manufactured by Sweco of Florence, Ky. or Kason Corporation of Millburn, N.J. Such a machine can sift the granulated sucralose particles through screens having varying sized pores, typically in a descending size order, in which each screen has a pore size slightly smaller than the pore size of the screen above it. From such a screening process, granules of sucralose separated into specific size ranges are obtained. One or more desired size range fractions may be isolated in this way, and one or more size range fractions outside the desired limits are typically produced as well. Some or all of this latter material may be recycled back into the fed for the compaction process, thereby reducing yield loss.

The granules are typically fractionated by size, typically by screening, with material outside the desired range being recycled into the chilsonation process. Granules according to the invention may be of any size. Typically they are between 30 and 2000 μm in size, by which is meant that at least 70 wt % of the granules are retained on a 30-μm screen and pass through a 2000-μm screen. Preferably, at least 80 wt %, more preferably at least 90 wt %, still more preferably at least 95 wt %, and most preferably substantially all of the granules are so passed and retained. More typically, the granules are within a more tightly controlled specific range whose limits depend upon the particular application for which the sucralose is intended.

The specific filters or screens are chosen on the basis of the desired size of the chilsonated sucralose particle. It is preferred that the final sucralose granules range from about 30 μm to about 2,000 μm in size. Typically the granules range from about 100 μm to about 800 μm in size, and more typically they range from about 150 to 500 μm in size. Granules of 150-300 μm size may be particularly useful for dry mix applications, while the 300-500 μm sized particles may be more suitable for chewing gums and tableting applications. In one embodiment of the invention, the granules are between 30 and 180 μm in size, preferably between 30 and 100 μm, and are especially useful in tableting applications.

As a result of the screening or other fractionation process, excessively small particles (“fines”) and excessively large ones (“overs”) are typically isolated. In one embodiment of the invention, instead of discarding the overs and fines, they are recycled back into the chilsonation process. Typically the overs and fines are mixed back in with, and fed into the compactor along with, the raw sucralose feed composition.

The final granulated and screened chilsonated sucralose particles are particularly suitable for tableting or mixing. The particles tend to be round or bead-like in shape and, as such, tend to minimize the dusting and clumping problems associated with the conventional micronized product. Therefore, the flow properties of chilsonated sucralose compositions according to the invention are improved over those of the neat product or the micronized product. Example 1 below provides detailed data regarding the flow properties of the product of this invention as compared with that of neat sucralose or micronized sucralose.

The granules produced by the methods of the invention are typically essentially solid, by which it is meant that they have few or no hollows or voids in them. The mechanical strength of the chilsonated sucralose product has also been found to be improved over the neat or micronized sucralose product. Mechanical strength of a particle is meant to describe the ability of the particle to retain its form while it is mixed and handled, for example in a packaging process. A particularly desirable feature for a high-intensity sweetener such as sucralose is the ability to retain good mixing and handling capabilities so as to avoid breaking apart and losing its form and effectiveness. The granulated sucralose of the invention has good mechanical strength, one beneficial result of which is that there is relatively little breakage and consequent formation of fines during handling, the presence of which can cause uneven sucralose distribution in formulations containing the product.

In addition to its good flow properties and mechanical strength, the chilsonated sucralose of the present invention also has surprisingly been shown to possess improved stability over the neat and micronized sucralose products presently used in the marketplace. Specifically, as set forth in the examples, neat product was able to maintain stability for only three days at 50° C., whereas the granulated material consistently maintained its stability for an additional day under the same stringent conditions. A one-day improvement in stability under these accelerated testing conditions is considered significant in the art for purposes of establishing a stability increase in a high-intensity sweetener. Example 1 below provides detailed data regarding the stability of the product of this invention as compared with that of neat sucralose or micronized sucralose.

Sucralose

Sucralose suitable for use in making the granulated product of this invention may be obtained following any of the procedures set forth in U.S. Pat. No. 4,362,869; U.S. Pat. No. 4,380,476; U.S. Pat. No. 4,801,700; U.S. Pat. No. 4,950,746; U.S. Pat. No. 5,470,969; and U.S. Pat. No. 5,498,709—all incorporated in this document by reference in all of these procedures, a final step in the synthesis of sucralose requires a deacylation followed by a crystallization of the sucralose. Typically, after completion of deacylation, the resulting sucralose is contacted with an ion exchange resin to convert the residual sodium methoxide to methanol. The ion exchange resin is then removed and the volatile solvents and reaction by-products are removed by co-distillation with water. The mixture is decolorized by contacting with activated carbon. The carbon is removed to provide decolorized sucralose solution suitable for crystallizing sucralose. The sucralose solution is concentrated to about 55 weight percent sucralose (at about 50° C.). The crystallization is performed by reducing the temperature to about 22° C. and adding about 2 percent sucralose seed crystals. The crystals that are formed are separated from the mother liquor by centrifugation to form a “wet cake”, which typically has a moisture content of about 3-5 wt %. The wet cake is typically then dried to a moisture content less than about 0.1 wt %.

Other Ingredients

Other ingredients such as carbohydrates, celluloses, gums, food acids, sweeteners such as nutritive and intense sweeteners, and flavorings may be incorporated in the feed prior to the compacting process to further improve functionality, quality, and stability. In many ingestible compositions, the use of an intense sweetener such as sucralose requires concurrent use of a bulking agent to provide acceptable bulk and texture to the final product. Many and various bulking agents (carriers, diluents, extenders) are known in the art, and may be incorporated into the feed along with the sucralose prior to compacting, so that the materials are compacted together. The amount and type of a particular bulking agent chosen for a specific composition must be such that it provides the specific bulk and texture required. In general, the selection of bulking agents is within the capabilities of those having ordinary skill in the art without undue experimentation.

Suitable carbohydrate bulking agents include sugars, sugar alcohols, hydrogenated hexoses, hydrogenated disaccharides, hydrogenated starch hydrolysates, soluble fibers (such as inulin, polydextrose, oligofructans, and others) and mixtures of these materials. Other suitable bulking agents include minerals such as calcium carbonate, talc, titanium dioxide, dicalcium phosphate, and the like.

Suitable sugar bulking agents include monosaccharides, disaccharides, and polysaccharides such as xylose, ribulose, glucose (dextrose), mannose, galactose, fructose (levulose), sucrose (table sugar), maltose, invert sugar, partially hydrolyzed starch and corn syrup solids, and mixtures of these materials. Mixtures of sucrose and corn syrup solids are particularly useful sugar bulking agents. Finally, suitable sugar alcohol bulking agents include sorbitol, xylitol, mannitol, galactitol, and mixtures of these materials.

One especially suitable bulking agent for use with the granulated sucralose of this invention is maltodextrin. In one exemplary embodiment of the invention, the presence of maltodextrin in a weight ratio to sucralose between about 400:1 and 800:1 provides a product having a sweetness per unit volume similar to that of table sugar, and also provides excellent stability.

### EXAMPLES

The advantageous properties of this invention can be understood by reference to the following examples. These are provided for the purposes of illustration and are not intended to limit the scope of the invention.

### Example 1

Three samples of sucralose powder were obtained. The samples were 1) sucralose wet cake (about 3 wt % moisture content) from a centrifuge, 2) neat sucralose (less than 0.1 wt % moisture content), and 3) a 1:3 blend by weight of these. Two other samples, designated 4) and 5), contained 30% and 50% of maltodextrin, respectively, blended with wet cake sucralose. Each of the samples was funneled into a lab scale 382 Chilsonator (The Fitzpatrick Company, Elmhurst, Ill.) having horizontal and vertical feed screws to meter, deaerate, and precompress the powder. The powder was compressed between two rolls in the chilsonator, with the resulting compacted product being fed to a mill. The blades of the mill broke the compacted product into smaller particles. The particles resulting from sample 3) were then screened using a Sweco sizing-sorting screening machine to produce granulated sucralose according to the following four size ranges.

>800 μm

400-800 μm

180-400 μm

<180 μm

These fractions were evaluated for bulk density, flowability and dissolution time at 20° C. Bulk density was measured using a cylinder of known volume. Tapped density was measured using the same bulk sample and cylinder by a tap machine tapping for 100 times. The cylinder was filled, recorded for the loose bulk volume, and the sample was then weighed, tapped, and recorded for the tapped volume. The flowability was measured as angle of repose. The rate of dissolution was measured by adding 2 g of sucralose to 98 mL of tap water with continuous, gentle stirring by a magnetic bar at medium speed. The time, to the nearest minute, required for complete disappearance of sucralose particles was recorded as the dissolution time. Table 1 summarizes the testing results for densities, flowability, and dissolution rate for the sieved fractions obtained from sample 3).

TABLE 1
Physical Properties of Granulated Sucralose

| | Granules | | | |
|---|---|---|---|---|
| | >800 μm | 400-800 μm | 180-400 μm | <180 μm |
| Loose bulk | 0.79 | 0.76 | 0.70 | 0.62 |
| density (g/mL) | | | | |
| Tapped bulk | 0.86 | 0.84 | 0.77 | 0.74 |
| density (g/mL) | | | | |
| Angle of repose | 28 | 28 | 33 | 40 |
| (degrees) | | | | |
| Dissolution time | <4 | <4 | <3 | <2 |
| (minutes) | | | | |

Note:
micronized sucralose has a bulk density ˜0.3-0.35 g/mL and angle of repose of >50 degrees.

Bulk and tapped densities ranged from 0.6-0.8 g/mL and 0.7-0.9 g/mL, respectively. These densities are comparable to those of dry food ingredients such as sugar and maltodextrin. Such ranges of density may help make in preparing uniform physical mixtures of the sucralose-containing granules of the invention with other food ingredients, and may help reduce packaging sizes and warehousing space.

Angle of repose values, as an indicator of flow properties, ranged from 28° to 40°, a significant improvement over that of micronized sucralose at 50° or higher. For most food or pharmaceutical powders, the angle of repose values range from 25° to 45°, with lower values indicating better flow characteristics. A good flow of granulated sucralose is helpful for efficient mixing and acceptable blending uniformity. In addition, improved flow may allow the granulated product to be packaged, either at the manufacturing site or during unitizing at customer production facilities, using high-speed packaging lines.

The data of Table 1 demonstrate that good flow properties, little dust, and high dissolution rate can be achieved for sucralose by a dry granulation process. Additionally, the data reveal that the physical properties of granulated sucralose could be modified and tailored for specific applications using compaction and particle size separation.

Given the improved flow properties and particle sizes, the various fractions are suitable for use in specific food and/or pharmaceutical applications. For example, both >800 μm and 400-800 μm can be easily handled and shipped for international purposes. The 180-400 μm product may be especially suitable for use in powdered products such as soft drinks and pharmaceutical dry mixes. The <180 μm product may be well suited for use in dry mixes or as a replacement for micronized sucralose.

Due to the high solubility of sucralose, the compressed granules even at >800 μm still exhibit good dissolution rate with mild stirring at room temperature. This property is important to many manufacturers of both dry powder mixes as well as liquid applications. For example, the carbonated soft drink industry currently packages dry agglomerated aspartame, another high-intensity sweetener, in high-speed form-fill-seal packaging lines for use in diet soft drink manufacture. Larger particles allow the product to be packaged at higher line speeds, but larger particles are more difficult for the end user to dissolve, and thus the poor dissolution properties of aspartame limit its maximum particle size to around 400 μm, thus limiting packaging line speed. Larger, rapidly dissolving particles of sweetener may therefore offer a significant operational benefit to primary purchasers of sucralose. In one embodiment of the invention, the sucralose-containing granules are between 800 and 2000 μm in size, and are especially suitable for packaging.

Table 2 shows the stability of the various sucralose samples described above in relation to Table 1, as well as for two chilsonated mixtures of sucralose with matodextrin. The data indicate an increase in the time to a one-unit drop in pH of a 10% by weight solution from 3 days for neat and micronized sucralose to 4-5 days for the chilsonated, and 7-8 days for the maltodextrin blended sucralose granules, and 6+ days for the wet cake (stopped at day 6).

TABLE 2
Comparison of Granulated Sucralose Stability at 50° C.

| | | pH Changes at various tested days | | | | | | | | |
|---|---|---|---|---|---|---|---|---|---|---|
| | | | | | | | | | | 50° C. |
| Sample | | | | | | | | | | Stability |
| Sample | Description | Day 0 | Day 2 | Day 3 | Day 4 | Day 5 | Day 6 | Day 7 | Day 9 | (Days) |
| 2 | Neat | −0.4 | −0.1 | −0.6 | −1.2 | | | | | 3 |
| 1 | Wet Cake | 0.1 | 0.1 | 0.1 | −0.1 | −0.2 | −0.2 | | | 6+ |
| 3 | Chilsonated, | −0.2 | −0.0 | −0.2 | −0.4 | −0.8 | −2.8 | | | 5 |
| | >800 μm | | | | | | | | | |
| 3 | Chilsonated, | 0.1 | 0.3 | −0.0 | −0.6 | −2.5 | | | | 4 |
| | 400-800 μm | | | | | | | | | |
| 3 | Chilsonated, | 0.4 | 0.4 | 0.3 | −0.6 | −2.5 | | | | 4 |
| | 180-400 μm | | | | | | | | | |
| 3 | Chilsonated, | 0.8 | 0.6 | 0.8 | −0.2 | −2.3 | | | | 4 |
| | <180 μm | | | | | | | | | |
| 4 | Chilsonated, | −0.2 | | | | −0.2 | | −0.3 | −1.5 | 7-8 |
| | 30% | | | | | | | | | |
| | Maltodextrin | | | | | | | | | |
| 5 | Chilsonated, | −0.0 | | | | −0.1 | | −0.6 | −2.6 | 7-8 |
| | 50% | | | | | | | | | |
| | Maltodextrin | | | | | | | | | |
| — | Micronized | | | | | | | | | 3 |
| | control | | | | | | | | | |

As can be seen from the data of Table 2, the granulated sucralose had improved stability relative to both the micronized and the neat sucralose samples. The presence of maltodextrin further improved stability.

The resulting granular sucralose produced from samples 1) and 3) had good flow properties, little dust formation, no visible evidence of static electric charge, and mechanical strength suitable for subsequent mixing processes. These samples also exhibited a heat stability at 50° C. of from 4-5 days and 6 or more days, respectively, versus three days for the product made from neat sucralose sample 2).

### Example 2

Accelerated Stability Study on Granulated/Chilsonated Sucralose

The following chilsonated sucralose samples A, B, and C were chilsonated as described in Example 1 above, for stability testing against a non-chilsonated control sample.

Sample A=30% wet cake/70% neat sucralose, chilsonated and screened to <180 μm size;

Sample B=30% wet cake/70% neat sucralose, chilsonated and screened to 400-800 μm size;

Sample C=30% wet cake/70% neat sucralose, chilsonated and screened to 400-800 μm size (repeat of Sample B);

Control=Neat sucralose, virgin feed, no chilsonation.

A 20-gram portion of each sample was placed in an 8-oz bottle and sealed for testing. Also, five 4-oz Whirl-Pak® bags (available from Nasco of Modesto, Calif.) were labeled for each sample and filled with 25 grams of neat product. Each of the five filled 4-oz Whirl-Pak bags for each sample was then sealed and placed into a separate 18-oz Whirl-Pak bag. The 18-oz bags were then sealed. Once all bags were prepared they were hung in a convection oven set at 50° C.

The samples were then monitored over a five-day period for changes in pH and appearance. On day zero, the contents of each 8-oz bottle were tested for these parameters and the results recorded. At 24 hours, and for each 24-hour period thereafter for the next 5 days, one bag from each sample was removed from the 50° C. oven and allowed to cool for 2 hours. At the end of the 2-hour period, the samples were moved into an 8-oz wide-mouth bottle and sealed. Description, color, consistency and odor were determined on the dry material. Additionally, the pH and solution color were determined on a 10% by weight solution of the sucralose in water, with the solution color compared with water.

The following four tables set forth the results from this study, and demonstrate an improved stability of the chilsonated product relative to that of the control. In the tables, “solution pH” was measured on a 10 wt % solution of the product in water, “water pH” was measured on the water used for making the solution, and pH change is equal to solution pH minus water pH.

Sample: A
Product Description: White, Loose, “Powdered Sugar” Odor

| Test | Day 0 | Day 1 | Day 2 | Day 3 | Day 4 | Day 5 | Day 6 |
|---|---|---|---|---|---|---|---|
| Date | Mar. 17, 2003 | Mar. 18, 2003 | Mar. 19, 2003 | Mar. 20, 2003 | Mar. 21, 2003 | Mar. 22, 2003 | |
| Product | White | — | White | White | white | Off-White | |
| Color | | | | | | | |
| Product | Loose | — | Loose | Loose | Loose | Loose | |
| Consistency | | | | | | | |
| Water pH | 6.1 | — | 5.9 | 6.0 | 5.9 | 6.1 | |
| Solution pH | 6.5 | — | 6.7 | 6.8 | 6.4 | 4.1 | |
| pH Change | 0.4 | — | 0.8 | 0.8 | 0.5 | −2.0 | |
| Solution | Clear | — | Clear | Clear | Clear | Yellow | |
| Color | | | | | | | |
| Product | Powdered | — | Powdered | Powdered | Powdered | Powdered | |
| Odor | Sugar | | Sugar | Sugar | Sugar | Sugar | |

Comments: Sample failed on Day 5. The final sample was off-white with a slight “powdered sugar” odor and formed a yellowish solution.

Sample: B
Product Description: White, Loose, Slight Sugary Odor

| Test | Day 0 | Day 1 | Day 2 | Day 3 | Day 4 | Day 5 | Day 6 |
|---|---|---|---|---|---|---|---|
| Date | Mar. 17, 2003 | Mar. 18, 2003 | Mar. 19, 2003 | Mar. 20, 2003 | Mar. 21, 2003 | Mar. 22, 2003 | |
| Product | White | — | White | White | White | light | |
| Color | | | | | | Yellow | |
| Product | Loose | — | Loose | Loose | Loose | Loose | |
| Consistency | | | | | | | |
| Water pH | 5.9 | — | 5.9 | 6.0 | 5.9 | 6.1 | |
| Solution pH | 6.2 | — | 5.9 | 5.9 | 5.5 | 4.1 | |
| pH Change | 0.3 | — | 0.0 | −0.1 | −0.4 | −2.0 | |
| Solution | Clear | — | Clear | Clear | Clear | Yellow | |
| Color | | | | | | | |
| Product | Slight | — | Slight | Slight | Slight | Slightly | |
| Odor | Sugary | | Sugary | Sugary | Sugary | Acidic | |

Comments: Sample failed on Day 5. The final sample was light yellow with a slight acidic odor and formed a yellow solution.

Sample: C
Product Description: White, Loose, Strong Sugary Odor

| Test | Day 0 | Day 1 | Day 2 | Day 3 | Day 4 | Day 5 | Day 6 |
|---|---|---|---|---|---|---|---|
| Date | Mar. 17, 2003 | Mar. 18, 2003 | Mar. 19, 2003 | Mar. 20, 2003 | Mar. 21, 2003 | Mar. 22, 2003 | |
| Product | White | — | White | White | White | Light | |
| Color | | | | | | Brown | |
| Product | Loose | — | Loose | Loose | Loose | Loose | |
| Consistency | | | | | | | |
| Water pH | 6.1 | — | 5.9 | 6.0 | 5.9 | 6.1 | |
| Solution pH | 6.6 | — | 6.2 | 6.1 | 5.3 | 3.2 | |
| pH Change | 0.5 | — | 0.3 | 0.1 | −0.6 | −2.9 | |
| Solution | Clear | — | Clear | Clear | Clear | Yellow | |
| Color | | | | | | | |
| Product | Strong | — | Strong | Strong | Strong | Slightly | |
| Odor | Sugary | | Sugary | Sugary | Sugary | Acidic | |

Comments: Sample failed on Day 5. The final sample was light yellow with a slight acidic odor and formed a yellow solution.

Sample: Control
Product Description: White, Loose, “Powdered Sugar” Odor

| Test | Day 0 | Day 1 | Day 2 | Day 3 | Day 4 | Day 5 | Day 6 |
|---|---|---|---|---|---|---|---|
| Date | Mar. 17, 2003 | Mar. 18, 2003 | Mar. 19, 2003 | Mar. 20, 2003 | Mar. 21, 2003 | | |
| Product | White | — | White | White | White | | |
| Color | | | | | | | |
| Product | Loose | — | Loose | Loose | Loose | | |
| Consistency | | | | | | | |
| Water pH | 6.1 | — | 5.9 | 6.0 | 5.9 | | |
| Solution pH | 6.0 | — | 6.0 | 5.8 | 4.7 | | |
| pH Change | −0.1 | — | 0.1 | −0.2 | −1.2 | | |
| Solution | Clear | — | Clear | Clear | Clear | | |
| Color | | | | | | | |
| Product | Powdered | — | Powdered | Powdered | Powdered | | |
| Odor | Sugar | | Sugar | Sugar | Sugar | | |

Comments: Sample failed on Day 4. The final sample was white with a “powdered sugar” odor.

Compositions made according to the invention have good flowability, low dusting, low static electric charge buildup, and good mechanical strength, thus making such a sucralose product well adapted for tableting or dry blending. In addition, in view of the ability of some embodiments of the process to allow for obtaining a wide particle size distribution range, compositions of this invention have utility in a variety of applications.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A composition comprising granules comprising sucralose, wherein the granules are between 30 and 2000 μm in size and have a moisture content between 0.5 and 1.5 wt %.

2. The composition of claim 1, wherein the moisture content is between 0.5 and 1.2 wt %.

3. The composition of claim 1, wherein the granules are between 100 and 800 μm in size.

4. The composition of claim 1, wherein the granules are between 30 and 180 μm in size.

5. The composition of claim 1, wherein the granules are between 800 and 2000 μm in size.

6. The composition of claim 1, wherein the granules further comprise one or more bulking agents selected from the group consisting of sugars, sugar alcohols, hydrogenated hexoses, hydrogenated disaccharides, hydrogenated starch hydrolysates, soluble fibers and mixtures of any of these.

7. The composition of claim 1, wherein the granules further comprise maltodextrin.

8. The composition of claim 1, wherein the granules further comprise sucrose.

9. The composition of claim 1, wherein the granules further comprise corn syrup solids.

10. A method for obtaining granules comprising sucralose, the method comprising the steps of:
A) providing a feed comprising sucralose, the feed having a starting moisture content selected such that the granules have a final moisture content between 0.5 and 1.5 wt %;
B) compacting the feed to form a compacted material; and
C) grinding the compacted material to form the granules.

11. The method of claim 10, wherein the granules are between 30 and 2000 μm in size.

12. The method of claim 10, wherein the final moisture content is between 0.5 and 1.2 wt %.

13. The method of claim 10, wherein the starting moisture content is between 0.5 and 3 wt %.

14. The method of claim 10, wherein the starting moisture content is between 0.7 and 1.5 wt %.

15. The method of claim 10, wherein the compacting is performed at a pressure between 1000 and 2000 psi.

16. The method of claim 10, wherein the compacting is performed with rollers.

17. The method of claim 16, wherein the rollers are textured.

18. The method of claim 16, wherein the rollers are polytetrafluoroethylene-coated rollers.

19. The method of claim 10, wherein the feed further comprises one or more bulking agents selected from the group consisting of sugars, sugar alcohols, hydrogenated hexoses, hydrogenated disaccharides, hydrogenated starch hydrolysates, soluble fibers and mixtures of any of these.

20. The method of claim 10, wherein the feed further comprises maltodextrin.

21. The method of claim 10, wherein the feed further comprises sucrose.

22. The method of claim 10, wherein the feed further comprises corn syrup solids.

23. The method of claim 10, wherein the feed further comprises corn syrup solids.

24. The method of claim 10, further comprising applying a vacuum to the feed prior to the compacting step.

25. The method of claim 10, further comprising:
D) dividing the material produced in step C) into one or more particle size range fractions.

26. The method of claim 25, further comprising:
E) recycling some or all of at least one of said one or more particle size range fractions into the feed.

27. The method of claim 25, wherein one of said one or more particle size range fractions consists of particles between 100 and 800 μm in size.

28. The method of claim 25, wherein one of said one or more particle size range fractions consists of particles between 800 and 2000 μm in size.

29. The method of claim 25, wherein one of said one or more particle size range fractions consists of particles between 30 and 180 μm in size.

30. Granules comprising sucralose, the granules made by a method comprising the steps of:
A) providing a feed comprising sucralose, the feed having a starting moisture content selected such that the granules have a final moisture content between 0.5 and 1.5 wt %;
B) compacting the feed to form a compacted material; and
C) grinding the compacted material to form the granules.
